(19) United States
(12) Patent Application Publication
(10) Pub. No.: US 2005/0131292 A1
(43) Pub. Date: Jun. 16, 2005

(54) Method and device for the automatic detection of motory disturbances in a test person

(75) Inventors: Michael Huss, Pohdaw (DE); Ekkehart Jenetzky, Ludwigsburg (DE)

Correspondence Address: ROTHWELL, FIGG, ERNST & MANBECK, P.C., 1425 K STREET, N.W., SUITE 800, WASHINGTON, DC, 20005, US

(73) Assignee: Charite, Universitaetsklinikum Medizinische Fakultaet der Humboldt-Universitaet zu Berlin, Berlin (DE)

(21) Appl. No.: 10/514,646

(22) PCT Filed: May 16, 2003
(86) PCT No.: PCT/DE03/01588

(30) Foreign Application Priority Data
May 16, 2002 (DE) 102 21 839.0

(51) Int. Cl. A61B 5/05

(52) U.S. Cl. 600/430

(57) ABSTRACT

The invention relates to a method and a device for the automatic detection of motor super-activity of a test person (1). With the help of a transmitting-/receiving means (2), microwave signals are transmitted and reflected microwave signals formed in the reflection on the test person (1) are received. With the help of an analysis of the Doppler frequency shift between the transmitted and received microwave signals, motor super-activity events in the test person (1) are detected. The frequency of the occurrence of motor super-activity events during a specified measuring time period is determined automatically and can be used for the diagnosis of psychiatric disorders in the test person (1).

The invention relates to a method and a device for the automatic detection of motor super-activity in a test person.

One of the most frequent disorders in child psychiatry is the so-called ADHD (“Attention Deficit Hyperactive Disorder”), which includes attention deficits, lack of impulse control as motor super-activity in children. Currently, ADHD is diagnosed primarily by using subjective questionnaire methods, which involves questioning the subjects suffering from ADHD as well as their relatives.

Acceleration-sensitive movement meters (“Akzellerometer”), where a plurality of measuring elements are attached to the body of the test person to record the movement of body parts, have been used to objectify the diagnostic methods. Attaching the measuring elements requires direct body contact with the test person and may lead to reactions in the test person which change the general movement behavior of the test person and thus severely limit the ability to evaluate the test person. Thus, methods on the basis of the “Akzellerometers” have not been able to gain acceptance in routine diagnostics.

Methods on the basis of electromyography (EMG) record only electrical muscle activity, which is not necessarily the same as actual movement. Because not each twitching of a muscle group automatically leads to a movement of a body part, this falsifies the measuring results. Therefore, these methods are also not suitable for the diagnosis of ADHD.

Another known method for recording motor super-activity events in a test person involves attaching optical reflectors to the body of the test person. With the help of a transmitting means, light signals are then transmitted into the direction of the test person to attempt to analyze the light reflected back by the reflectors on the test person's body and obtain information about the motor super-activity of the test person. This method also has the disadvantage already mentioned earlier, i.e., that the reflectors attached to the test person's body influence the test person's regular movement. Furthermore, detecting the light reflected by the various reflectors on the test person's body requires a high effort with respect to technical equipment.

Thus, the problem to be solved by the invention is to provide an improved method and an improved device of the type described initially, which enables a reliable automatic detection of motor super-activity of a test person in a simple manner without influencing the remaining movements of a test person in the performance of the method and the application of the device.

In accordance with the invention, this problem is solved with a method in accordance with the independent claim 1 and a device in accordance with the independent claim 8.

The invention comprises particular the idea to use microwave signals for the automatic detection of motor super-activity events in a test person by using the Doppler frequency shift that occurs between the transmitted microwave signals and the received microwave signals reflected on the test person as an indicator for the presence of a motor super-activity event. Compared to the state of the art, the use of the Doppler effect in combination with microwaves has the significant advantage that the motor super-activity events can be detected at the test person's body without the attachment of specific measuring means or reflectors. In this way, an extensive objectification of the detection of the motor super-activity events is achieved. The objectification of the measurement also improves the diagnostic options in the determination of psychiatric disorders related to motor super-activity of the test person as well as in reviewing the effect of drug therapy following the diagnosis of such disorders.

Furthermore, the detection of any motor super-activity events in the test person who is within the transmitting cone of the emitted microwave signals enables a greater precision of the measurement compared to the known “Akzellometer” test method because the “twitching” of individual muscle groups is not falsified and registered as a motor super-activity event.

The invention furthermore has the advantage that for the first time, it is possible to use technical means to detect super-activity events emanating in any body part of the test person. In this way, the non-contact measurement can be objectively quantified globally. Thus, for routine procedures, subjective influences, which play a significant role in the questionnaires generally used today, can be avoided.

A useful development of the new method provides that with the help of a distance measuring means, a repeated measurement of the distance between the test person and the distance measuring means and/or objects in the area of the transmitting cone and the distance measuring means is executed automatically in timely correlation to the transmitting of the microwave signals. In this way, falsified measuring signals in the detection of the motor super-activity of the test person, which are the result of other objects in motion such as another person, for example, can be eliminated in the area of the transmitting cone if a distance that does not correspond to the usual distance between the test person and the distance measuring means is determined in a timely correlation during the detection of a Doppler frequency shift resulting from a movement of the test person.

In a useful development of the invention, a way of measuring the distance which can be executed with simple means and rules out any damage to the test person is achieved in that additional microwave signals or optical signals are transmitted and reflected portions of the transmitted additional microwave/optical signals are received and analyzed to measure the distance with the help of the distance measuring means.

For the further objectification and discriminating effect of the measuring results, it may be provided in an advantageous embodiment of the invention that the reflected microwave signals are detected over a specified period of time in the course of resting phases and stimulated activity phases of the test person, with electronic information about a start and an end of the resting phases and the stimulated activity phases being recorded automatically by the evaluation device.

In a preferred development of the invention, a way of stimulating a test person is achieved with simple means in that light signals are transmitted with the help of a photo stimulation lamp to stimulate the activity phases.

In a useful development, an improved diagnostic option is created in that at least during part of the specified time period, a measurement of the test person's brain waves is performed with the help of a brain wave measuring device, with a timely electronic correlation of the brain wave signals detected by the brain wave measuring device and the received reflected microwave signals as well as the determined Doppler frequency shifts being performed automatically. The defined measuring conditions for measuring the brain waves also create measuring parameters for the detection of the super-activity events, which ensures the comparability of the measuring results for different measurements.

A useful development of the invention may provide that by means of the recorded brain waves an error correction of the determined frequency of occurrence of the motor super-activity events is performed automatically in the specified time period with the help of the evaluation device taking account the timely correlation. With the occurrence of specific brain wave signals, which can be associated with reactions of the test person not related to a motor super-activity movement of the test person, the Doppler frequency shifts recorded at the same time as said brain wave signals can be ruled out automatically as indicators for motor super-activity events.

A preferred embodiment of the invention provides that an automatic error correction of the recorded brain wave signals is performed with the help of the evaluation device, taking into account the timely correlation with the help of the determined Doppler frequency shifts.

The dependent device claims list the aforementioned advantages in connection with the respective related dependent method claim.

The invention is explained in greater detail in the following with the example of an embodiment referring to illustrations, which show:

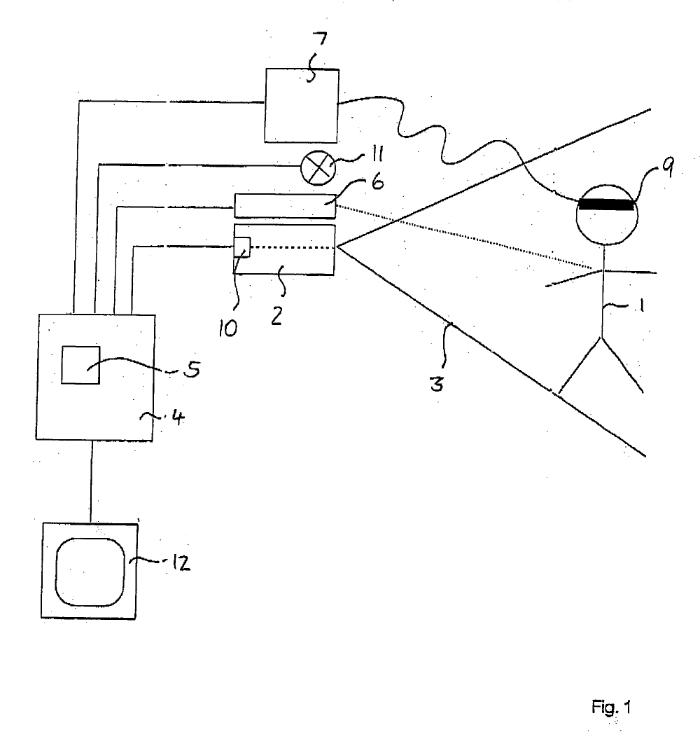

FIG. 1 a schematic representation of an arrangement to record motor super-activity events in a test person;

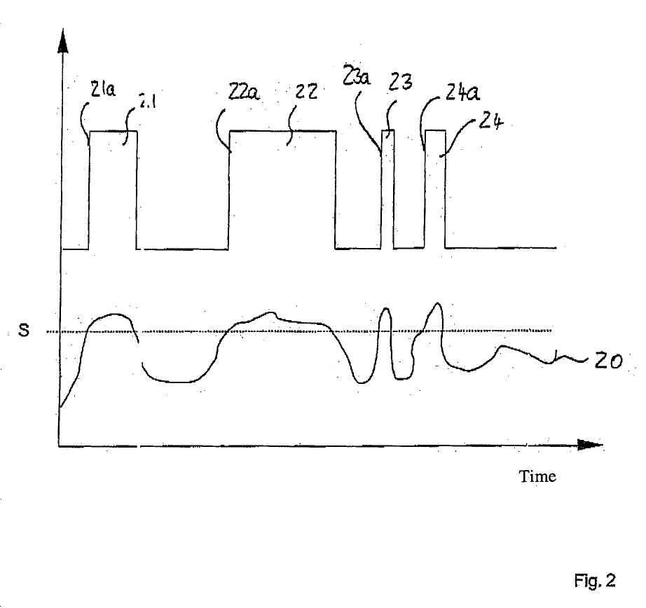

FIG. 2 a graphic representation of the example of a signal curve; and

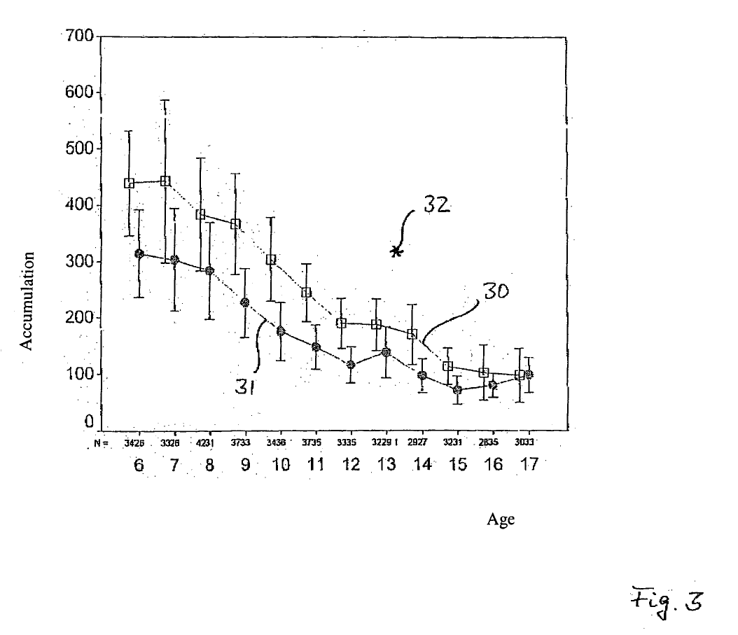

FIG. 3 a graphic representation of the averaged frequency of motor super-activity event signals in test persons of various ages.

FIG. 1 shows a schematic representation of an arrangement for the automatic detection of motor super-activity events for a test person 1. With the help of a transmitting-/receiving means 2, microwave signals within a transmitting cone 3 where the test person 1 is located are transmitted. Transmitted microwaves that are reflected at the test person 1 can be received with the help of the transmitting-/receiving means 2 as reflected microwaves. The transmitting-/receiving means 2 is preferably developed as a combined transmitting-/receiving device (transceiver), however, separate devices for the trasmitting and receiving of microwaves may also be provided.

If a reflected microwave signal was reflected at a body part of the test person making a movement, the reflected microwave signal has, in comparison to the transmitted microwave signals, a Doppler frequency shift that is the result of the relative movement between the body part and the transmitting-/receiving means 2. For all received, reflected microwave signals, the Doppler frequency shift relative to the transmitted microwave signals is detected with the help of a Doppler evaluation means 10. In the embodiment according to FIG. 1, the Doppler evaluation means 10 is arranged in the sending-/receiving means 2. The measuring principle of the Doppler radar which forms the basis of the described method is well known as such and used in various Doppler radar devices; therefore, said measuring principle does not require any detailed explanation here.

FIG. 2 shows a graphic representation of the example of a measuring curve 20 for the course of the Doppler frequency shift of the received microwave signals measured continually during a specified measuring period. The information concerning the Doppler frequency shift of the received microwave signals are transmitted by the transmitting-/receiving means 2 to an evaluation means 4 (see FIG. 1). With the help of the evaluation means 4, which has a microprocessor means 5 to process the transmitted electronic information for the Doppler frequency shift, the measured curve 20 is processed to generate digitalized signals according to the principle of an analogue-digital conversion. According to FIG. 2, rectangle impulses 21, 22, 23 and 24 are generated whenever the curve 20 exceeds a threshold value S, with the threshold value S corresponding to a preset Doppler frequency shift that indicates an actual motor super-activity event.

In the subsequent determination of the frequency of occurrence of motor super-activity events, the number of accelerating flanks 21a, 22a, 23a and 24a of the rectangle impulses 21, 22, 23 and 24 is determined automatically with the help of the control- and evaluation device 4. For this purpose, the electronic results of the analog-digital-conversion are analyzed. The frequency of the motor super-activity events within the specified measuring period as determined in this manner provides information for the test person's diagnostics, such as the presence of ADHD (“attention deficit hyperactive super-activity”). For diagnostic conclusions, the frequency value determined for a test person can be compared to a calibration curve that was determined for a large number of the test persons. With the help of a comparison of the frequency measuring results for a test person at various points in time, it is also possible to analyze the effect of drug therapy for the test person.

FIG. 3 shows a graphic representation of mean values of the determined frequency of motor super-activity events for male (30) and female (31) test persons depending on the age of the test person. The number N of test persons for whom the measuring results were taken into account to determine the represented mean values is shown above the value for the age along the X-axis. A measuring point 32 is far above the average values for a 13-year old test person, meaning that in said case, there are clear indications of an ADHD.

In the following, additional details of the arrangement according to FIG. 1 are described. An essential element of the transmitting-/receiving means 2 is a high frequency oscillator that is used as a transmitter and works in a microwave range of 9.35 GHz. The emitted total performance of the transmitting means is approximately 1 mW, although it can be adjusted to the respective application case to obtain on the one hand a sufficient signal-noise ratio, and on the other hand, rule out with certainty any damage to the test person as a result of microwave radiation. In connection with microwave ovens, 5 mW per cm² are considered as completely harmless for humans. In the transmitting-/receiving means 2 used here, the response limit for movements of the test person 1 is at least 6 cm/[illegible] to a maximum of 80 km/h. A further optimization to increase the sensitivity of the transmitting-/receiving means 2 may be provided, but may increase the production costs of the transmitting-/receiving unit 2, if applicable.

The high frequency oscillator used here, for example, has a cone-shaped emission characteristic of about +/−20 degrees. However, the receiving means detects an opening angle of approximately +/−60 degrees up to a distance of approximately 5 to 8 meters. Generally, a test person 1 is located in a distance of 1 to 2 meters from the transmitting-/receiving means 2. These parameters may be adapted to various ambient conditions depending on the application case. To that end, one skilled in the art can take advantage of the experiences in the area known as microwave sensory.

Furthermore, according to FIG. 1, a distance meter 6 is provided as well. This may be any distance meter that is known as such and is suitable for use in experiments with living things, in particular due to the source of radiation being used. Preferably, a distance meter with an optical radiation source with low radiation intensity is used. With the help of the distance meter 6, the distance between the distance meter 6 and the test person and/or another object in the area of the transmitting cone 6 is measured during the specified measuring period. The results of the distance measurement are transmitted from the distance meter 6 to the evaluation means 4 so that respective associated distance measuring values are available as additional measuring information in connection with the measuring series recorded with the help of the transmitting-/receiving means 2. In a comparison of different measuring series, the distance measuring values thus can be used as additional criteria for evaluation or comparison, for example to take into account a distance-dependent measuring value detection of the transmitting-/receiving means 2.

With the help of the results of the distance measurement, signals can be automatically discarded as errors in the evaluation of the digitalized signals 21-24 (see FIG. 2) if at the time of the signal, a distance measured with the help of the distance meter 6 deviates from the usual distance between the distance meter 6 and the test person 1, which leads to a discontinuity and points to an object other than the test person 1. This may be, for example, someone checking the measurement and walking between the distance meter 6 and the test person 1. So as to be able to associate the measured distance signals with the digitalized signals 21 to 24, the transmitting-/receiving means 2 and the distance meter 6 have to work on a common time basis. This can be ensured, for example, with the help of a common interval timer (not shown) in the evaluation means 4 or in one of the two devices, i.e., the transmitting-/receiving means 2 or the distance meter 6. Said interval timer may be a conventional electronic timing pulse generator as it is used to generate time impulses in pulsed measurements. It should be pointed out that the measuring of the motor super-activity events on the test person 1 with the help of the transmitting-/receiving means 2 principally can be performed independent of the measurements with the help of the distance meter 6. However, the use of the distance meter 6 is to reduce the number of falsified signals that are erroneously interpreted as motor super-activity events. Furthermore, in accordance with FIG. 1, a brain wave measuring device 7 is coupled to the control- and evaluation device 4. With the help of the brain wave measuring means 7, a standard EEG (EEG—electroencephalography) is recorded. For this purpose, the brain wave measuring device 7 is coupled to electrodes 9 arranged on the body of the test person 1 through of one or a plurality of lines 8. A timely correlation of the brain waves measured with the help of the brain wave measuring means 7 and the received reflected microwave signals and/or the Doppler frequency shifts detected therefrom is performed in the evaluation means 4. This makes it possible, when measuring the brain waves with the help of the brain wave measuring means 7 and measuring the motor super-activity events with the help of the transmitting-/receiving means 2, to rule out mutual falsified signals. This is performed in the evaluation means 4 with the help of an automatic error correction. Thus, this makes it possible, for example, to rule out specific signals of the digitalized signals 21-24 as indicators for a motor super-activity event if it is determined from the course of the measured brain waves that a movement of a body part of the test person 1 cannot have occurred. Vice versa, the digitalized signals 21-24 can also offer conclusions abut erroneous brain wave signals.

Thus, a combination of the transmitting-/receiving means 2 and the brain wave measuring device 7 enables a reduction of the frequency of errors in the detection of the motor super-activity events. However, this is an improved embodiment of the device for the detection of motor super-activity events in a test person 1 because the method for determining the motor super-activity events can also be performed without a combination with the brain wave measuring means 7.

In connection with the measuring of brain waves, the test person 1 will generally pass through resting phases and stimulated activity phases in a specified manner. Stimulated activity phases may comprise a hyperventilation of the test person 1, for example. A photo stimulation lamp 11 (see FIG. 1), which is coupled with the control- and evaluation device 4, is provided to stimulate the activity phases. An effective photo stimulation lamp is a conventional lamp for emitting light, which can be operated in impulse operation. It has been experienced that the prescribed standardized measuring conditions for the measuring of brain waves, in particular the specified sequence of resting phases and stimulated activity phases, support the comparability and/or statistical accuracy of the measuring results for the automatic detection of the frequency of motor super-activity events for the test person 1, with in particular the measuring results being detected in the resting phases being evaluated for diagnostics. In the automatic determination of the frequency, the control- and evaluation means 4 automatically takes into account electronic information concerning the timely association of resting phases and stimulated phases of activity during the specified measuring period.

The control- and evaluation device 4, including the micro processor means 5, may be developed as separate unit, as is shown schematically in FIG. 1 by means of the block representation, or it can be integrated in one of the measuring devices, i.e., the transmitting-/receiving means 2 or the distance meter 6 or the brain wave measuring means 7. The detected measuring signals and/or the results determined in the evaluation, for which a respective time information, i.e. in particular information concerning the time of day and/or the day and/or the duration of the measurement, can be generated in the described measuring arrangement, can be shown on a display means 12. In another embodiment, the display means 12 can be combined with the transmitting-/receiving means 2 or the distance meter 6 or the brain wave measuring means 7. The measuring devices, i.e., the transmitting-/receiving means 2 and/or the distance meter 6 and/or the brain wave measuring means 7, in as far as they are provided in the respective embodiment, are provided as single devices, or they may be combined in one device. The single devices or the combination devices are preferably developed as portable devices, which for the user simplifies the handling in diagnostics use.

The advantages related to the method and the device for the detection of the motor super-activity events are also obtained without a combination with the distance meter 6 and/or the brain wave measuring device 7. The essential advantages include that the entire physical motor skills of the test person 1 can be detected. Only the actual movements of the test person 1 are detected, but not the contraction of the muscles. There is no actual contact whatsoever between the measuring device in form of the transmitting-receiving means 2 being used and the body of the test person 1, which could influence the usual movement sequence of the test person and lead to a falsification of the measuring result. The described measuring device is easy to handle and thus suitable for routine use to detect measuring results.

The characteristics of the invention disclosed in the preceding description, the claims and the illustration may be of significance for the realization of the invention in its various embodiments either individually or also in any combination.

1. Method for the automatic detection of motor super-activity in a test person (1) with the method comprising the following steps:
Transmitting microwave signals in a transmitting cone (3) by means of a microwave transmission means (2) for a specified period of time:
Receiving reflected microwave signals that are formed in the reflection of the transmitted microwave signals on a test person located in the area of the transmitting cone (3), by means of a microwave receiving means (2);
Automatic determination of Doppler frequency shifts between the transmitted microwave signals and reflected microwave signals resulting from motor super-activity events in the test person (1) by means of a Doppler evaluation means (10); and
Automatic determination of a frequency of occurrence of motor super-activity events in the specified time period depending on the determined Doppler frequency shifts, by means of an evaluation means (4).

2. Method in accordance with claim 1, characterized in that in timely correlation to the transmitting of the microwave signals, a repeated measurement of the distance between the test person (1) and the distance measuring means (6) and/or objects in the area of the transmitting cone (3) and the distance measuring means (6) is performed with the help of a distance measuring means (6).

3. Method in accordance with claim 2, characterized in that additional microwave signals or optical signals are transmitted to measure the distance with the help of a distance measuring means (6), and reflected parts of the transmitted additional microwave signals or optical signals are received and evaluated.

4. Method in accordance with claim 1, characterized in that the reflected microwave signals are detected during the specified time period in the course of resting phases and of stimulated activity phases of the test person (1), with electronic information about a start and an end of the resting phases and the stimulated activity phases being recorded automatically by the evaluation device (4).

5. Method in accordance with claim 4, characterized in that light signals are transmitted with the help of a photo stimulation lamp to stimulate the activity phases.

6. Method in accordance with claim 1, characterized in that at least during a part of the specified time period, a measurement of the test person's brain waves is performed with the help of a brain wave measuring device (7), with an electronic timely correlation of the brain wave signals detected by the brain wave measuring device (7) being performed automatically for the received reflected microwave signals as well as the determined Doppler frequency shifts.

7. Method in accordance with claim 6, characterized in that with the help of the evaluation device (1) and taking into account the timely correlation by means of the detected brain wave signals, an error correction of the determined frequency of occurrence of the motor super-activity events during the specified time period is performed automatically.

8. Method in accordance with claim 6, characterized in that with the help of the evaluation device (4) and taking into account the timely correlation, an error correction is performed automatically with the help of the automatically determined Doppler frequency shifts of an error correction of the detected brain wave signals.

9. Device for the automatic detection of motor super-activity in a test person (1), with the device having the following characteristics:
a microwave transmitting means (2) to transmit microwave signals in a transmitting cone (3) for a specified time period;
a microwave receiving means (2) to receive reflected microwave signals that are formed when the transmitted microwave signals reflect on a test person (1) located in the range of the transmitting cone (3);
a Doppler evaluation means (10) for the automatic determination of Doppler shifts between the transmitted microwave signals and reflected microwave signals as a result of motor super-activity events in the test person (1); and
an evaluation device (4) for the automatic determination of a frequency of the occurrence of motor super-activity events depending on the Doppler frequency shifts determined during the specified time period.

10. Device in accordance with claim 9, characterized by a local memory means for the electronic storage of information related to the determined Doppler frequency shifts and/or the determined frequency of the occurrence of the motor super-activity events.

11. Device in accordance with claim 9, characterized in that the device is developed as a portable handheld device.

12. Device in accordance with claim 9, characterized in that the microwave transmitting means and the microwave receiving means are integrated in one transmitting-/receiving means (2).

13. Device in accordance with claim 9, characterized in that the microwave transmitting means and the microwave receiving means for the timely coordination of the transmitting of the microwave signals and the receiving of the reflected microwave signals with a brain wave measurement in the test person (1) with the help of a brain wave measuring device (7) are coupled to a common interval timer unit.

14. Device in accordance with claim 13, characterized in that a photo stimulation lamp for transmitting light signals is coupled to the brain wave measuring device (7).

15. Use of a device in accordance with one of the claims 9 claim 9 for the automatic detection of motor super-activity events in a test person, in particular for a diagnostic examination to determine ADHD super-activity (“Attention Deficit Hyperactive Super-activity”) depending on determined Doppler frequency shifts of microwave signals.
